(19) United States
(12) Patent Application Publication
(10) Pub. No.: US 2025/0049631 A1
(43) Pub. Date: Feb. 13, 2025

(54) SYSTEMS AND METHODS FOR VIBRATION ANESTHESIA

(71) Applicant: Stephen COMITE, New York, NY (US)

(72) Inventor: Stephen COMITE, New York, NY (US)

(21) Appl. No.: 18/799,096

(22) Filed: Aug. 9, 2024

Related U.S. Application Data
(60) Provisional application No. 63/518,912, filed on Aug. 11, 2023.

(51) Int. Cl. A61H 23/02

(52) U.S. Cl. CPC A61H 23/0263; A61H 2201/0107; A61H 2201/0153; A61H 2201/0157; A61H 2201/1215; A61H 2201/1657; A61H 2201/169; A61H 2201/5023

(57) ABSTRACT

A system and method for reducing pain in a subject includes providing a device with a hollow body having at least two ends, a vibratory motor mounted in the hollow body configured for transmitting vibrations of variable frequency and/or voltage, a switch on the body to turn the vibratory motor on and off, and a tip. The tip includes a proximal section removably coupled to one end of the body, a distal section extending forwardly from the body, and a contact element on the distal section, wherein the contact element has an open geometric shape.

### FIELD OF THE DISCLOSURE

The present disclosure relates to systems and methods for vibrational anesthesia.

### BACKGROUND OF THE DISCLOSURE

Many people fear needles and that can impact and decrease their willingness to get vaccines and undergo medical procedures, treatments and tests. A substantial percentage of patients are needle-phobic. This affects children as well as adults. For example, some geriatric patients are hesitant to get a flu shot. Human immunodeficiency virus (HIV) patients might delay being tested.

Effective means to decrease needle pain include local analgesia, distracting the patient, and competing with the nerves. Topical analgesics can reduce or eliminate needle pain but cost between US$12 and US$150 per patient use and some require prescriptions. In addition, topical analgesics require prolonged application times or can cause vasoconstriction decreasing venipuncture success.

Accordingly, there is a need for a device method that allows for the reduction of the pain associated with hypodermic needle sticks.

### SUMMARY OF THE DISCLOSURE

The present disclosure provides a system and method for reducing pain in a subject.

In some embodiments, the present disclosure provides a device for reducing pain in a subject, comprising a hollow body having at least two ends, a vibratory motor mounted in the hollow body configured for transmitting vibrations of variable frequency and/or voltage, a switch or a button on the body to turn the vibratory motor on and off, and a tip. In some embodiments, the tip includes a proximal section removably coupled to one end of the body, a distal section extending forwardly from the body, and a contact element on the distal section, wherein the contact element has an open geometric shape.

In some embodiments, the tip is disposable. In some embodiments, the hollow body has a longitudinal axis, wherein the proximal section of the tip has a longitudinal axis that is parallel with the longitudinal axis of the hollow body and wherein the distal section of the tip has a longitudinal axis that is at an angle in the range of from 0° to 90° relative to the longitudinal axis of the hollow body. In some embodiments, the contact element has a longitudinal axis that is at an angle in the range of from 0° to 90° relative to the longitudinal axis of the hollow body. In some embodiments, the device further comprises a hinge that is coupled to the proximal and distal sections of the tip, the hinge being adjustably configured to change the angle between the longitudinal axis of the tip and the longitudinal axis of the hollow body. In some embodiments, the vibratory motor transmits vibrations at a frequency in the range of from 2.5 Hz to 1000 Hz. In some embodiments, the switch is a variable dimmer switch configured to control the frequency and/or voltage of the vibrations transmitted. In some embodiments, the vibratory motor is turned on and off by the button and wherein the device further comprises one or more vibration toggle buttons to control the frequency and/or voltage of the vibrations transmitted at discrete vibrational levels. In some embodiments, the device comprises a rechargeable battery and is rechargeable. In some embodiments, the device comprises a recharging port. In some embodiments, the recharging port is a USB A, USB 2.0 B, USB 3.0, USB C, USB 2.0 micro, USB 3.0 micro, USB mini, lightning type port. In some embodiments, the device further comprises a charger stand that generates inductive current, and wherein the rechargeable battery is configured to recharge with inductive current. In some embodiments, the open geometric shaped contact element has an inner diameter that is in the range of from 1 cm to 50 cm. In some embodiments, the contact element is configured for transferring the vibrations from the motor to an anesthesia zone on an area of skin of the subject when the tip touches the skin, thereby producing a vibrational analgesia effect and reducing pain in the zone. In some embodiments, the contact element further comprises a plurality of nodes.

In some embodiments, the present disclosure provides a method for reducing pain in a subject, comprising providing a device that includes a hollow body having at least two ends, a vibratory motor mounted in the hollow body configured for transmitting vibrations of variable frequency and/or voltage, a switch or a button on the body to turn the vibratory motor on and off, and a tip having a proximal section removably coupled to one end of the body, a distal section extending forwardly from the body, and a contact element on the distal section, wherein the contact element has open geometric shape, positioning the contact element on an anesthesia zone, turning on the vibratory motor to produce vibrations from the motor that are transferred to the anesthesia zone, and maintaining the contact element on the anesthesia zone for a time period prior to and during an application of a needle to the anesthesia zone, whereby the vibrations are configured to produce a vibrational analgesia effect on the subject so as to reduce pain in the subject.

In some embodiments, the tip is disposable. In some embodiments, the hollow body has a longitudinal axis, wherein the proximal section of the tip has a longitudinal axis that is parallel with the longitudinal axis of the hollow body and wherein the distal section of the tip has a longitudinal axis that is at an angle in the range of from 0° to 90° relative to the longitudinal axis of the hollow body. In some embodiments, the contact element has a longitudinal axis that is at an angle in the range of from 0° to 90° relative to the longitudinal axis of the hollow body. In some embodiments, the device further comprises a hinge that is coupled to the proximal and distal sections of the tip, the hinge being adjustably configured to change the angle between the longitudinal axis of the tip and the longitudinal axis of the hollow body. In some embodiments, wherein the vibratory motor transmits vibrations at a frequency in the range of from 2.5 Hz to 1000 Hz. In some embodiments, the switch is a variable dimmer switch configured to control the frequency and/or voltage of the vibrations transmitted. In some embodiments, the vibratory motor is turned on and off by the button and wherein the device further comprises one or more vibration toggle buttons to control the frequency and/or voltage of the vibrations transmitted at discrete vibrational levels. In some embodiments, the device comprises a rechargeable battery and is rechargeable. In some embodiments, the device comprises a recharging port. In some embodiments, the recharging port is a USB A, USB 2.0 B, USB 3.0, USB C, USB 2.0 micro, USB 3.0 micro, USB mini, lightning type port. In some embodiments, the device further comprises a charger stand that generates inductive current, and wherein the rechargeable battery is configured to recharge with inductive current. In some embodiments, the open geometric shaped contact element has an inner diameter that is in the range of from 1 cm to 50 cm. In some embodiments, the contact element is maintained on the anesthesia zone for a time period in the range of from 5 to 1200 seconds. In some embodiments, the distal section of the tip is configured for transferring the vibrations from the motor to an anesthesia zone on an area of skin of the subject when the tip touches the skin. In some embodiments, the contact element further comprises a plurality of nodes.

Any combinations of the various embodiments and implementations disclosed herein can be used. These and other aspects and features can be appreciated from the following description of certain embodiments and the accompanying drawings and claims.

### BRIEF DESCRIPTION OF THE DRAWINGS

FIG. 1 is perspective view of a device according to one or more embodiments.

FIG. 2 is a side view of the device according to one or more embodiments.

FIG. 3 is a side view of the device according to one or more embodiments.

FIG. 4 is a cross sectional view of the device according to one or more embodiments.

FIG. 5 is a front view of the device according to one or more embodiments.

FIG. 6 is a front rear of the device according to one or more embodiments.

FIG. 7 is a perspective view of the device according to one or more embodiments, showing the tip removed from the hollow body.

FIG. 8 is a side view of the device according to one or more embodiments, showing the tip removed from the hollow body.

FIG. 9 is a side view of the device according to one or more embodiments.

FIG. 10 is a side view of the device according to one or more embodiments.

FIGS. 11a-11e are a side view of the device according to one or more embodiments.

FIG. 12 the device according to one or more embodiments being used to reduce pain in a subject's arm.

FIG. 13 is perspective view of a device according to one or more embodiments.

FIG. 14 is a side view of the device according to one or more embodiments.

FIG. 15 is a side view of the device according to one or more embodiments.

FIG. 16a is a side view of the device according to one or more embodiments.

FIG. 16b is a cross sectional view of the device according to one or more embodiments.

FIG. 17 is a front view of the device according to one or more embodiments.

FIG. 18 is a front rear of the device according to one or more embodiments.

FIG. 19 is a perspective view of the device according to one or more embodiments, showing the tip removed from the hollow body.

FIG. 20 is a side view of the device according to one or more embodiments, showing the tip removed from the hollow body.

FIG. 21a is a perspective view of the tip of the device according to one or more embodiments.

FIGS. 21b-21e are side views of tips of the device according to one or more embodiments.

FIG. 22a is a side view of the tip of the device according to one or more embodiments.

FIG. 22b is a side view of the tip of the device according to one or more embodiments.

FIG. 23a is a perspective view the charger stand of the device according to one or more embodiments.

FIG. 23b is a side view of a of the device according to one or more embodiments.

FIG. 24 the device according to one or more embodiments being used to reduce pain in a subject's arm.

### DETAILED DESCRIPTION OF CERTAIN EMBODIMENTS OF THE DISCLOSURE

The present disclosure is directed to a system and method for reducing pain in a subject.

In some embodiments, the present disclosure provides a device for reducing pain in a subject, comprising a hollow body having at least two ends, a vibratory motor mounted in the hollow body configured for transmitting vibrations of variable frequency and/or voltage, a switch or button on the body to turn the vibratory motor on and off, and a tip. In some embodiments, the tip includes a proximal section removably coupled to one end of the body, a distal section extending forwardly from the body, and a contact element on the distal section, wherein the contact element has an open geometric shape.

In some embodiments, the tip is disposable. In some embodiments, the hollow body has a longitudinal axis, wherein the proximal section of the tip has a longitudinal axis that is parallel with the longitudinal axis of the hollow body and wherein the distal section of the tip has a longitudinal axis that is at an angle in the range of from 0° to 90° relative to the longitudinal axis of the hollow body. In some embodiments, the contact element has a longitudinal axis that is at an angle in the range of from 0° to 90° relative to the longitudinal axis of the hollow body. In some embodiments, the device further comprises a hinge that is coupled to the proximal and distal portions of the tip, the hinge being adjustably configured to change the angle between the longitudinal axis of the tip and the longitudinal axis of the hollow body. In some embodiments, the vibratory motor transmits vibrations at a frequency in the range of from 2.5 Hz to 1000 Hz. In some embodiments, the switch is a variable dimmer switch configured to control the frequency and/or voltage of the vibrations transmitted. In some embodiments, the vibratory motor is turned on and off by the button and wherein the device further comprises one or more vibration toggle buttons to control the frequency and/or voltage of the vibrations transmitted at discrete vibrational levels. In some embodiments, the device comprises a rechargeable battery and is rechargeable. In some embodiments, the device comprises a recharging port. In some embodiments, the recharging port is a USB A, USB 2.0 B, USB 3.0, USB C, USB 2.0 micro, USB 3.0 micro, USB mini, lightning type port. In some embodiments, the device further comprises a charger stand that generates inductive current, and wherein the rechargeable battery is configured to recharge with inductive current. In some embodiments, the open geometric shaped contact element has an inner diameter that is in the range of from 1 cm to 50 cm. In some embodiments, the contact element is configured for transferring the vibrations from the motor to an anesthesia zone on an area of skin of the subject when the tip touches the skin, thereby producing a vibrational analgesia effect and reducing pain the zone. In some embodiments, the contact element further comprises a plurality of nodes.

In some embodiments, the present disclosure provides a method for reducing pain in a subject, comprising providing a device that includes a hollow body having at least two ends, a vibratory motor mounted in the hollow body configured for transmitting vibrations of variable frequency and/or voltage, a switch or a button on the body to turn the vibratory motor on and off, and a tip having a proximal section removably coupled to one end of the body, a distal section extending forwardly from the body, and a contact element on the distal section, wherein the contact element has open geometric shape, positioning the contact element on an anesthesia zone, turning on the vibratory motor to produce vibrations from the motor that are transferred to the anesthesia zone, and maintaining the contact element on the anesthesia zone for a time period prior to and during an application of a needle to the anesthesia zone, whereby the vibrations are configured to produce a vibrational analgesia effect on the subject so as to reduce pain in the subject.

In some embodiments, the tip is disposable. In some embodiments, the hollow body has a longitudinal axis, wherein the proximal section of the tip has a longitudinal axis that is parallel with the longitudinal axis of the hollow body and wherein the distal section of the tip has a longitudinal axis that is at an angle in the range of from 0° to 90° relative to the longitudinal axis of the hollow body. In some embodiments, the contact element has a longitudinal axis that is at an angle in the range of from 0° to 90° relative to the longitudinal axis of the hollow body. In some embodiments, the device further comprises a hinge that is coupled to the proximal and distal portions of the tip, the hinge being adjustably configured to change the angle between the longitudinal axis of the tip and the longitudinal axis of the hollow body. In some embodiments, wherein the vibratory motor transmits vibrations at a frequency in the range of from 2.5 Hz to 1000 Hz. In some embodiments, the switch is a variable dimmer switch configured to control the frequency and/or voltage of the vibrations transmitted. In some embodiments, the vibratory motor is turned on and off by the button and wherein the device further comprises one or more vibration toggle buttons to control the frequency and/or voltage of the vibrations transmitted at discrete vibrational levels. In some embodiments, the device comprises a rechargeable battery and is rechargeable. In some embodiments, the device comprises a recharging port. In some embodiments, the recharging port is a USB A, USB 2.0 B, USB 3.0, USB C, USB 2.0 micro, USB 3.0 micro, USB mini, lightning type port. In some embodiments, the device further comprises a charger stand that generates inductive current, and wherein the rechargeable battery is configured to recharge with inductive current. In some embodiments, the open geometric shaped contact element has an inner diameter that is in the range of from 1 cm to 50 cm. In some embodiments, the contact element is maintained on the anesthesia zone for a time period in the range of from 5 to 1200 seconds. In some embodiments, the contact element is configured for transferring the vibrations from the motor to an anesthesia zone on an area of skin of the subject when the tip touches the skin. In some embodiments, the contact element further comprises a plurality of nodes.

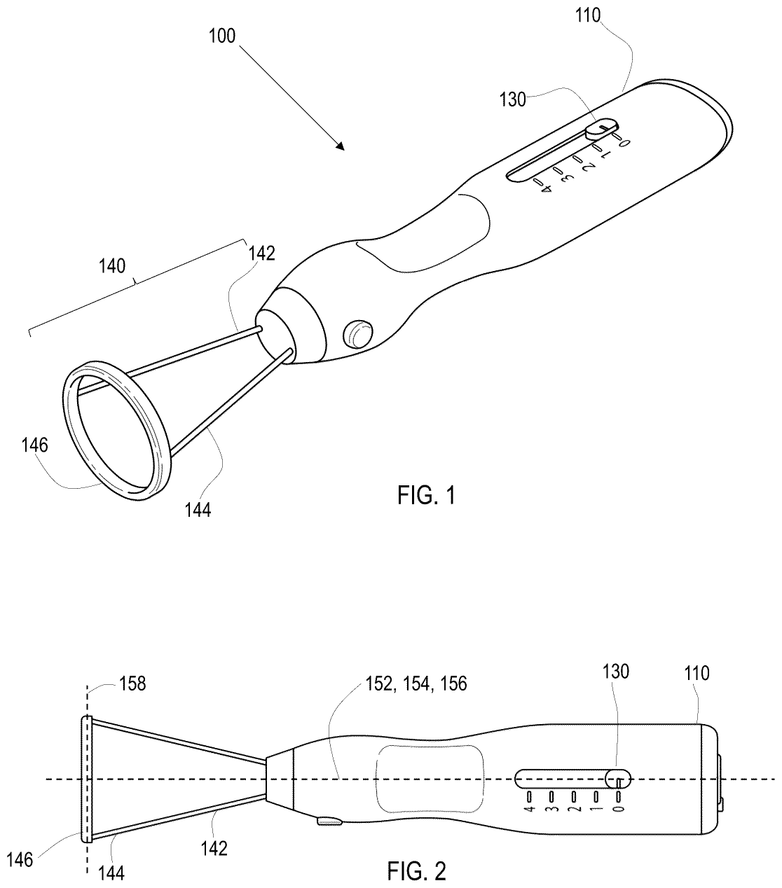

A perspective view of a device according to one or more embodiments of the present disclosure is shown in FIG. 1. Device 100 includes a hollow body 110, a vibratory motor (shown as 120 in FIG. 4), a switch 130 and a tip 140. The vibratory motor 120 is mounted in the hollow body configured for transmitting vibrations of variable and/or discrete various frequencies and/or voltages.

The tip 140 includes a proximal section 142 and a distal section 144. The distal section of the tip 144 includes a contact element 146.

The hollow body has a longitudinal axis 152. The proximal section of the tip has a longitudinal axis 154. The distal section of the tip has a longitudinal axis 156. The contact element has a longitudinal axis 158.

FIG. 2 shows a top view of the device 100 of one or more embodiments. In the embodiment shown in FIG. 2, the longitudinal axis of the hollow body 152, the longitudinal axis of the proximal section of the tip 154 and the longitudinal axis of the distal section of the tip 156 are all parallel and coaxial with each other. The longitudinal axis 158 of the contact element is at an angle of 90° relative to the longitudinal axis of the hollow body.

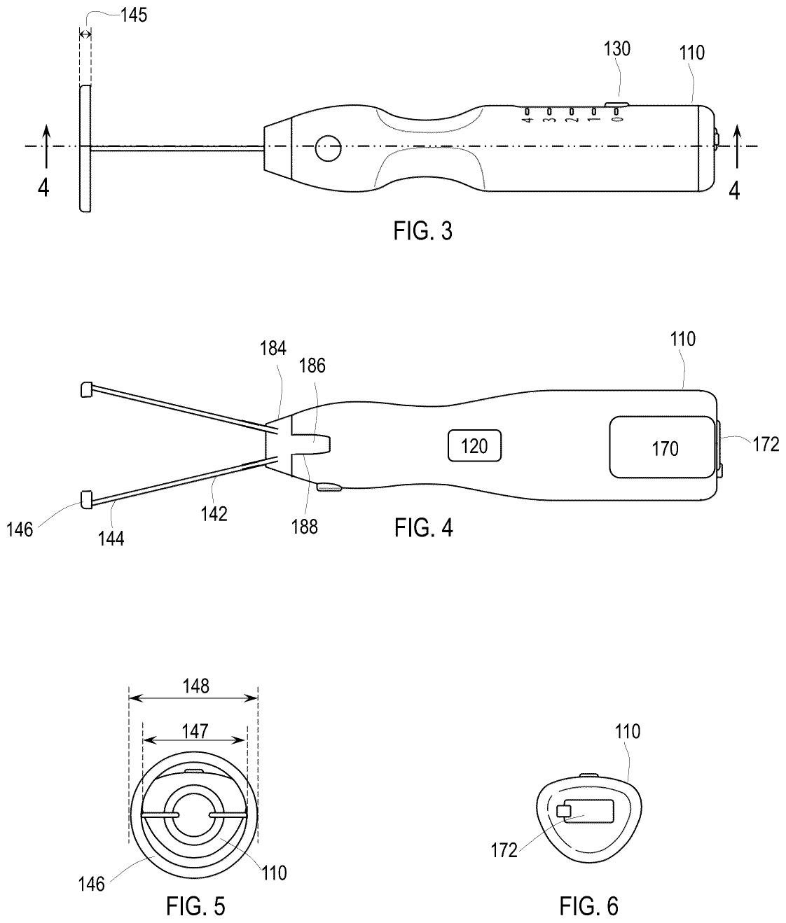

FIG. 3 is a side view of the device 100 of one or more embodiments. FIG. 4 shows a section of device 100 taken along the line 4-4 of FIG. 3. The vibratory motor 120 and a rechargeable battery 170 are located within the hollow body 110.

FIG. 5 shows a front view of the device 100 of one or more embodiments. Contact element 146 has an inner diameter 147 and an outer diameter 148. FIG. 6 shows a rear view of the device 100 of one or more embodiments. Device 100 includes a recharging port 172. In some embodiments (not shown), the battery 170 is not rechargeable, for example a single use, disposable, or primary battery. In these embodiments, there is no recharging port 172. In some embodiments (not shown), there is no recharging port 172 and instead the device can be recharged via an indictive current provided by a charger stand (not shown).

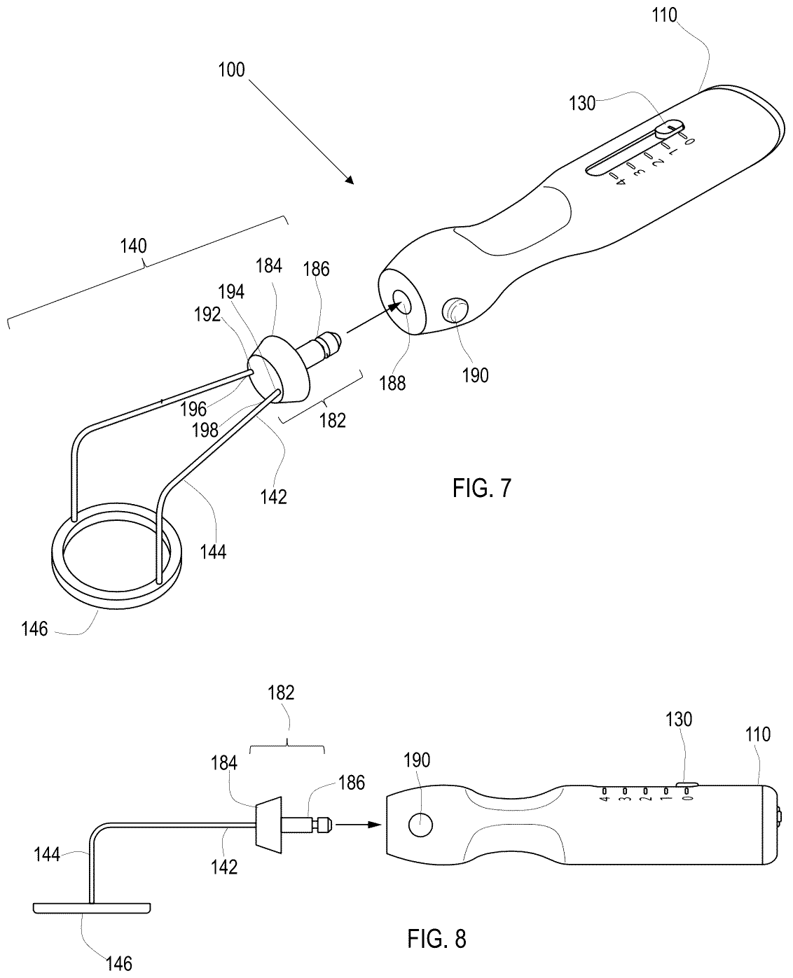

FIG. 7 is a perspective view of the device 100 according to one or more embodiments of the present disclosure. FIG. 8 is a side view of the device 100 according to one or more embodiments of the present disclosure. In these embodiments, the tip 140 is shown separated from the hollow body 110. Connector element 182 includes distal section 184, and a proximal section 186. Proximal section 186 is configured as a male end. Hollow body 100 includes a female connector element 188 and a release button 190. To attach/engage the tip 140 with the hollow body 110, a user will engage push the male end of the proximal section of the connector element 186 into the female connector element 188 on the hollow body 110. To disengage the 140 from the hollow body 110, a user will push the release button 190 which will allow the connector element 182 to be removed from the hollow body 110.

Distal section of connector element 184 includes two or more holes/female connection sections in it, labeled as features 192 and 194 in FIG. 7. Proximal section 142 of the tip includes two or more prongs, labeled as features 196 and 198 in FIG. 7. In some embodiments, the tip 140 is disposable. In these embodiments, once used, the tip 140 can be removed from the connector element 182 by disengaging the two prongs 196 and 198 from holes 192 and 194. Then a new tip 140 can be replaced by engaging the two prongs 196 and 198 on a new tip into holes 192 and 194. In some embodiments, not shown, more than two holes and two prongs are used, such as 3, 4, 5, or more. In some embodiments, as shown in FIG. 8, the longitudinal axis of the distal section of the tip 156 is about 90° relative to the longitudinal axis of the hollow body 152 and the length of distal section 144 of tip 140 is of a sufficient length so that the contact element 146 can make contact with the skin of a user without the hollow body 110 also making contact with the skin of a user.

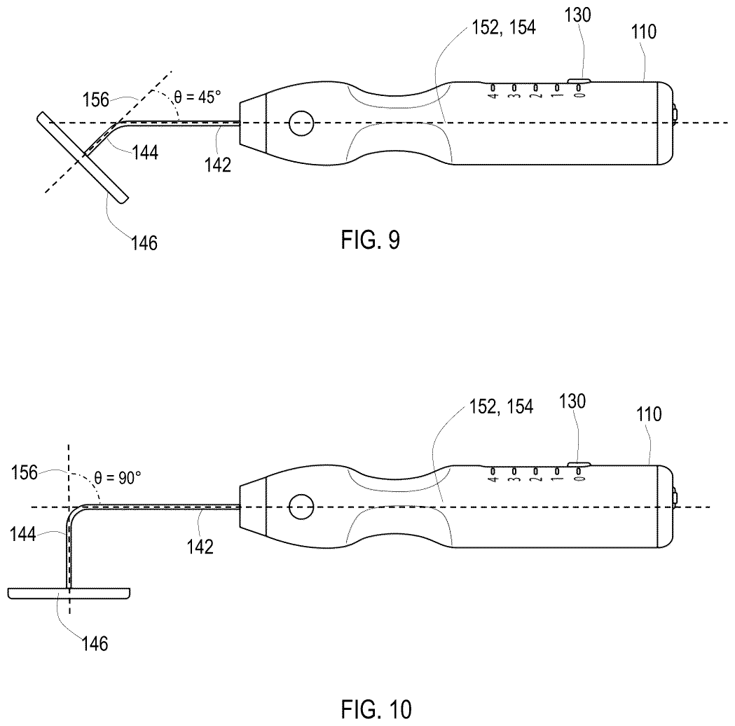

In some embodiments, the longitudinal axis of the distal section of the tip 156 is not parallel and coaxial with the longitudinal axis of the hollow body 152 and the longitudinal axis of the proximal section of the tip 154, as shown in FIGS. 9 and 10. In FIG. 9, the longitudinal axis of the distal section of the tip 156 is at an angle θ of about 45° with respect to the longitudinal axis of the hollow body 152. In In FIG. 10, the longitudinal axis of the distal section of the tip 156 is at an angle θ of about 90° with respect to the longitudinal axis of the hollow body 152.

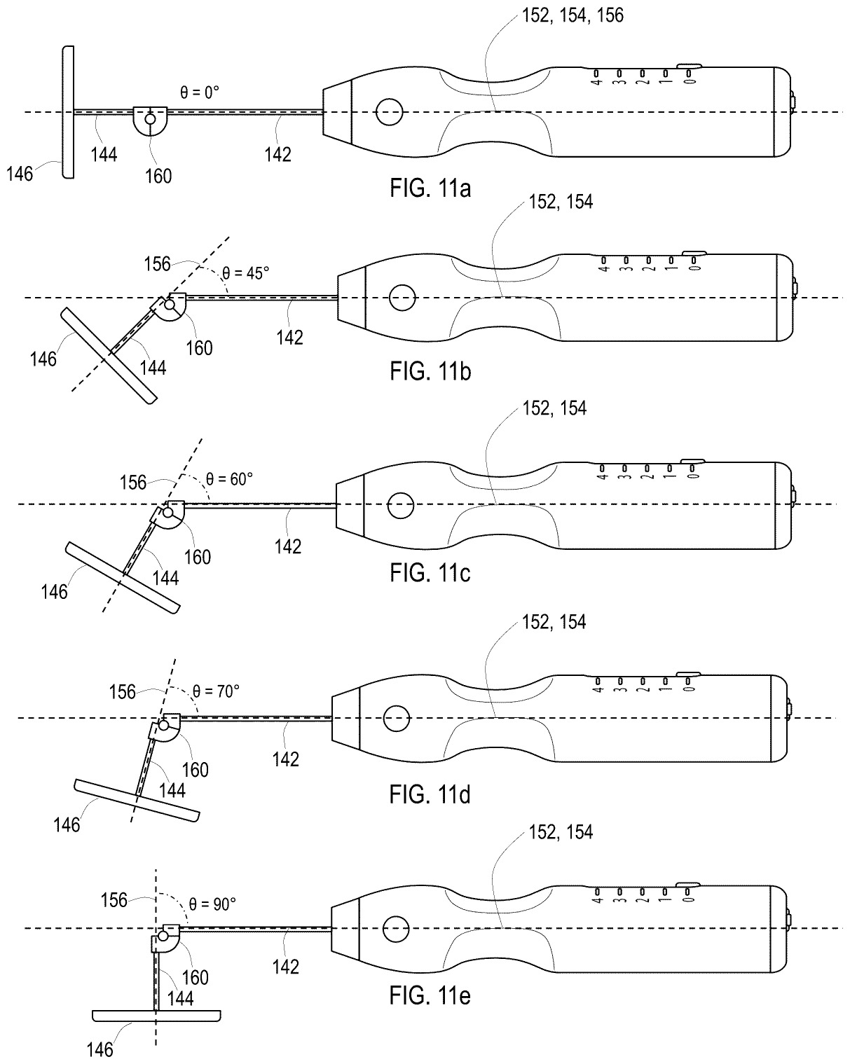

In some embodiments, the device 100 also includes a hinge 160, as shown in FIGS. 11a-11e. Hinge 160 is coupled to the proximal section of the tip 142 and the distal section of the tip 144. Hinge 160 can be adjusted to change the angle between the longitudinal axis of the tip 156 and the longitudinal axis of the hollow body 152. In some embodiments the hinge 160 allows the angle between the longitudinal axis of the tip 156 and the longitudinal axis of the hollow body 152 to be adjusted to discrete angles, in other embodiments, the hinge 160 allows the angle between the longitudinal axis of the tip 156 and the longitudinal axis of the hollow body 152 to be adjusted along a continuum of angles (not discrete).

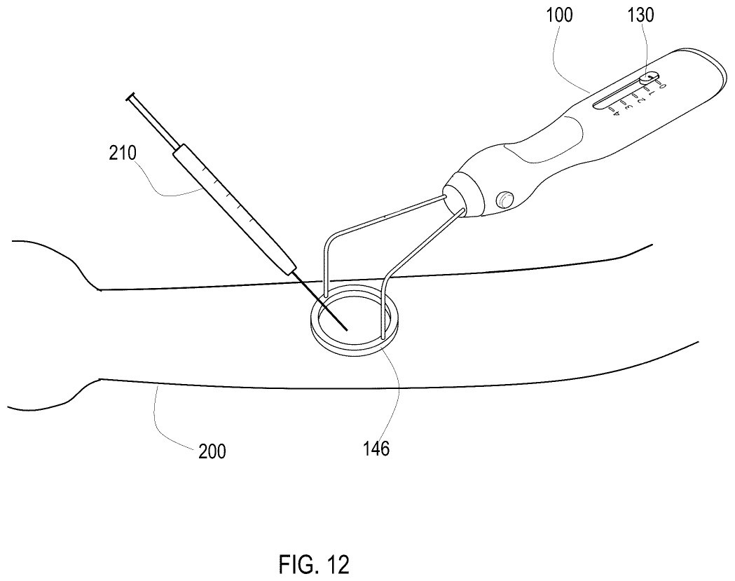

FIG. 12 shows the device 100 of one or more embodiments in use. The contact element 146 of device 100 will be positioned on an area of subject's arm 200. The vibratory motor 120 will be turned on by using the switch 130. This will produce vibrations that are transferred from the contact element 146 to the subject's arm 200 in an anesthesia zone. The contact element 146 is maintained on the arm 200 for a time period prior to and during an application of a needle 210. In some embodiments, as shown in FIG. 12, the needle is injected into an area of the skin that is within the open geometric shaped contact element. The vibrations produce a vibrational analgesia effect on the arm 200 so as to reduce pain in the subject.

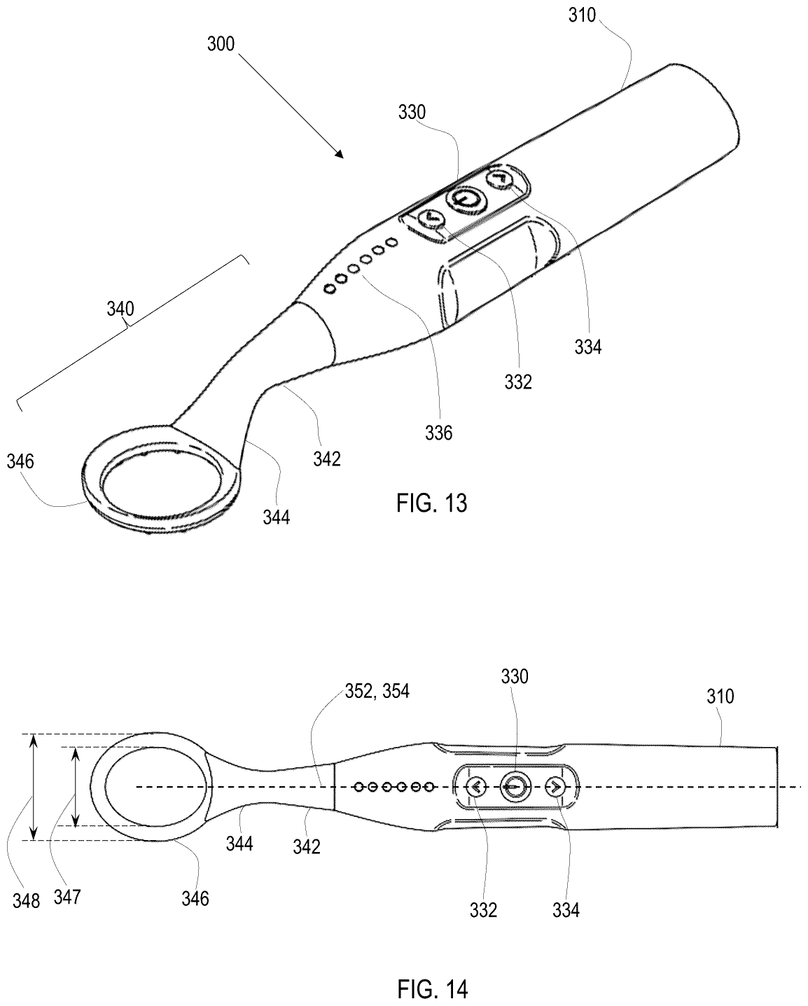

A perspective view of a device according to one or more embodiments of the present disclosure is shown in FIG. 13. Device 300 includes a hollow body 310, a vibratory motor (shown as 320 in FIG. 16b), an on/off button 330, one or more vibration toggle buttons 332, 334, and a tip 340. The vibratory motor 320 is mounted in the hollow body configured for transmitting vibrations of variable and/or discrete various frequencies and/or voltages. In some embodiments, the hollow body 310 also includes one or more LED indicator lights 336. LED indicator lights 336 can show what vibrational level the device is set to. For example, if the if the vibrational level is set to the second lowest setting, two LED lights can be illuminated.

The tip 340 includes a proximal section 342 and a distal section 344. The distal section of the tip 344 includes a contact element 346.

The hollow body has a longitudinal axis 352. The proximal section of the tip has a longitudinal axis 354. The distal section of the tip has a longitudinal axis 356. The contact element has a longitudinal axis 358.

FIG. 14 shows a top view of the device 300 of one or more embodiments. In the embodiment shown in FIG. 14, the longitudinal axis of the hollow body 352, and the longitudinal axis of the proximal section of the tip 154 are parallel and coaxial with each other.

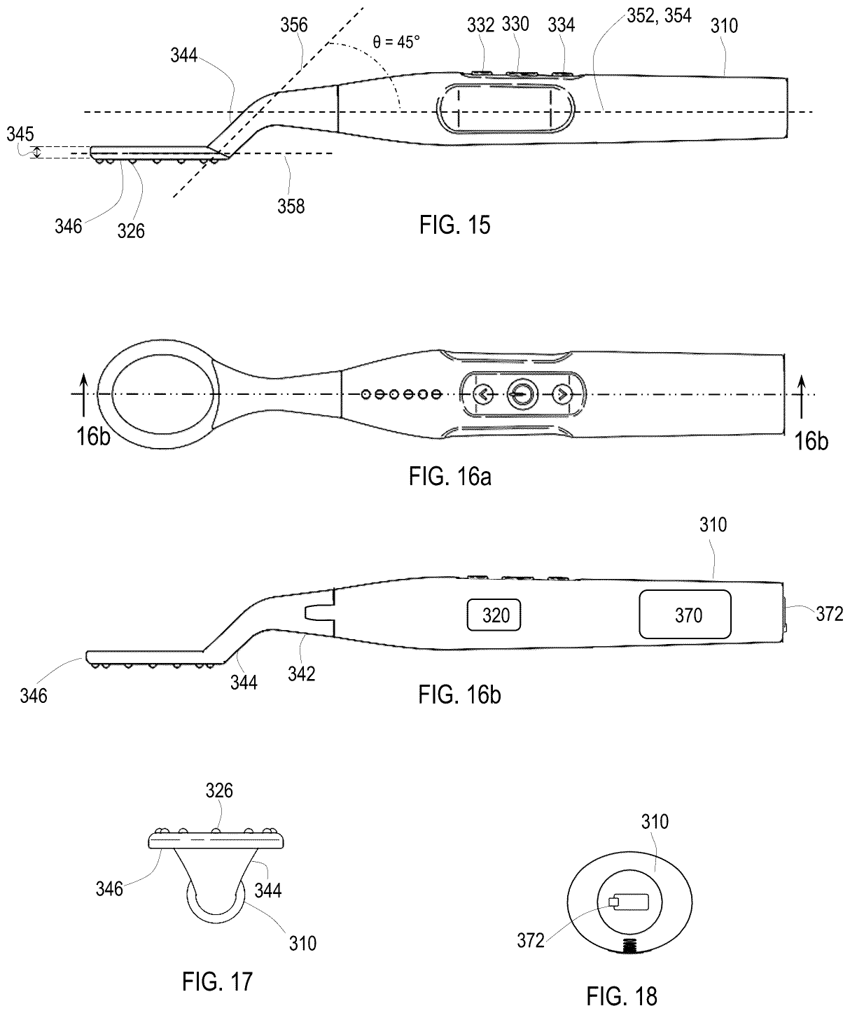

FIG. 15 is a side view of the device 300 of one or more embodiments. As shown in FIG. 15, in some embodiments, the longitudinal axis of the distal section of the tip 356 is not parallel and/or coaxial with longitudinal axis of the hollow body 352 and/or longitudinal axis 356 of the distal section of the tip, but rather at an angle. In FIG. 15, the angle is about 45°, but other angles in the range of from 0° to 90° are also suitable. In FIG. 15, the longitudinal axis 358 of the contact element is parallel to, but not coaxial with, the longitudinal axis of the hollow body 352. In other embodiments, the longitudinal axis 358 of the contact element can be at other angles in the range of from 0° to 90° relative to the longitudinal axis of the hollow body 352. In some embodiments, the longitudinal axis 358 of the contact element is parallel to, but not coaxial with, the longitudinal axis of the hollow body 352, and the length of the distal section of the tip 344 is of a sufficient length so that the contact element 346 can make contact with the skin of a user without the hollow body 310 also making contact with the skin of a user.

In some embodiments, the contact element 346 comprises a plurality of nodes 326.

FIG. 16a shows a top view of the device 300 of one or more embodiments. FIG. 16b shows a section of device 300 taken along the line 16b-16b of FIG. 16a. The vibratory motor 320 and a rechargeable battery 370 are located within the hollow body 310.

FIG. 17 shows a front view of the device 300 of one or more embodiments. Contact element 346 has an inner diameter 347 and an outer diameter 348. FIG. 18 shows a rear view of the device 300 of one or more embodiments. In some embodiments, device 300 includes a recharging port 372. In some embodiments (not shown), the battery 370 is not rechargeable, for example a single use, disposable, or primary battery. In these embodiments, there is no recharging port 372. In some embodiments, there is no recharging port 372 and instead the device can be recharged via an indicative current provided by a charger stand.

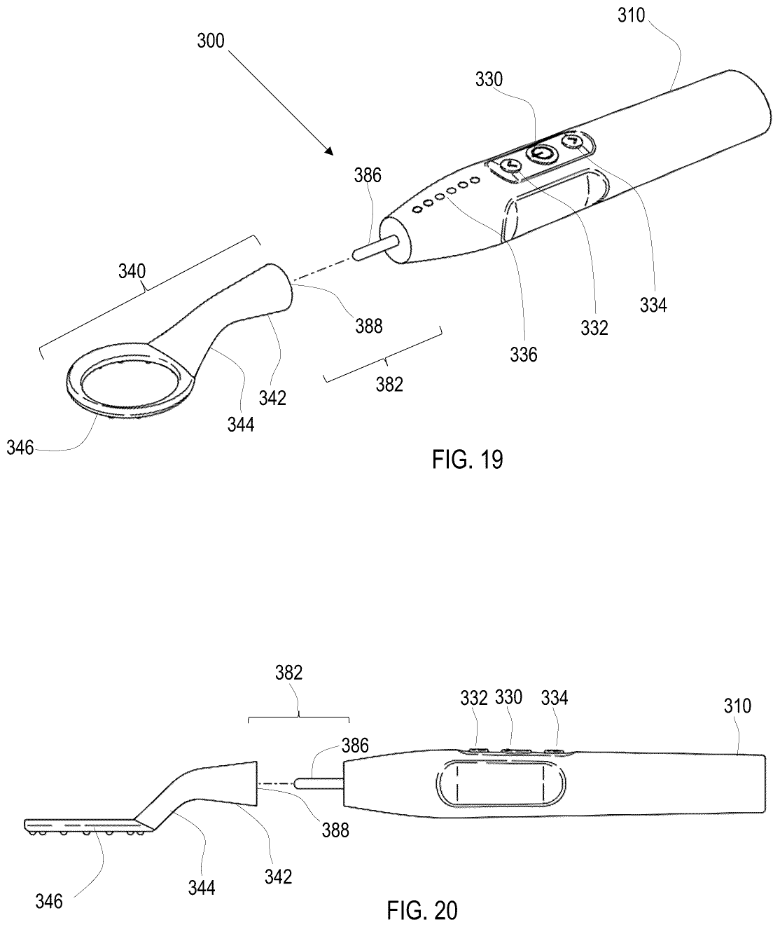

FIG. 19 is a perspective view of the device 300 according to one or more embodiments of the present disclosure. FIG. 20 is a side view of the device 300 according to one or more embodiments of the present disclosure. In these embodiments, the tip 340 is shown separated from the hollow body 310. Connector element 382 includes distal section 388, and a proximal section 386. Proximal section 386 is configured as a male end and is in connected to hollow body 310. The tip 340 includes a female connector element 388. To attach/engage the tip 340 with the hollow body 310, a user will engage push the male end of the proximal section of the connector element 386 into the female connector element 388 on the tip 340. In some embodiments, the connector element 382 is configured so that when fully engaged, the user can hear and/or feel a click to indicate that the connection is secure. To disengage the tip 340 from the hollow body 310, a user will pull the tip 340 from the hollow body 310 which will allow disengagement. In some embodiments, he connector element 382 is configured so that when the user successfully disengages the tip 340 from the hollow body 310, they can hear and/or feel a click to indicate that the connection is full disengaged. In some embodiments (not shown), there is a release button similar to described above in FIGS. 8 and 9.

In some embodiments, the longitudinal axis of the distal section of the tip 356 is not parallel and coaxial with the longitudinal axis of the hollow body 352 and the longitudinal axis of the proximal section of the tip 354, as shown in FIG. 15. In FIG. 15, the longitudinal axis of the distal section of the tip 356 is at an angle θ of about 45° with respect to the longitudinal axis of the hollow body 352.

In some embodiments, the device 300 also includes a hinge (not shown), similar to hinge 160 shown in FIGS. 11a-11e. The hinge can be coupled to the proximal section of the tip and the distal section of the tip and can be adjusted to change the angle between the longitudinal axis of the tip and the longitudinal axis of the hollow body. In some embodiments the hinge allows the angle between the longitudinal axis of the tip and the longitudinal axis of the hollow body to be adjusted to discrete angles, in other embodiments, the hinge allows the angle between the longitudinal axis of the tip and the longitudinal axis of the hollow body to be adjusted along a continuum of angles (not discrete).

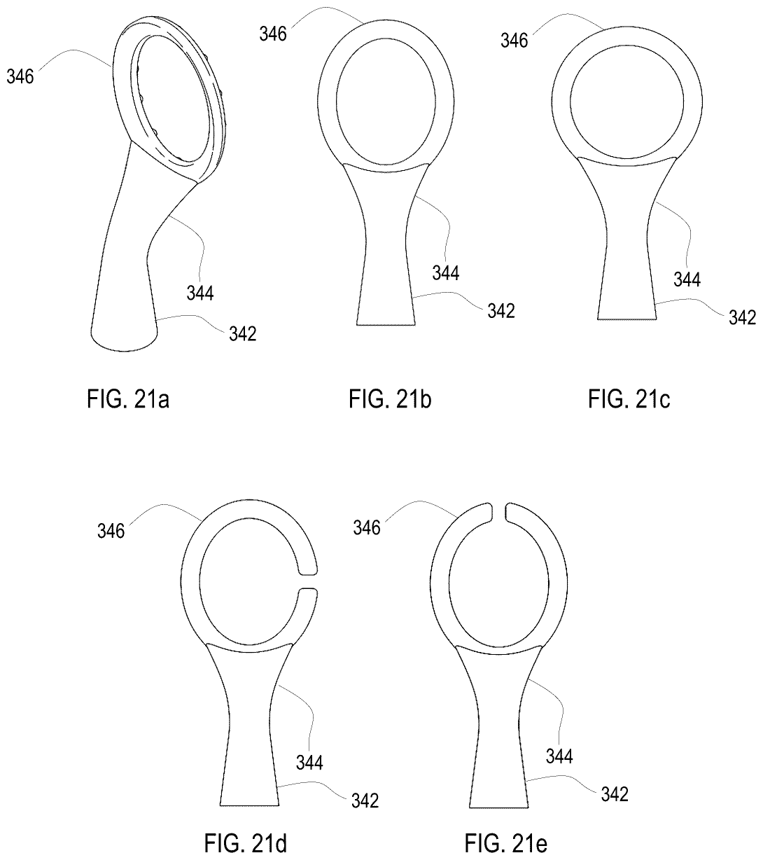

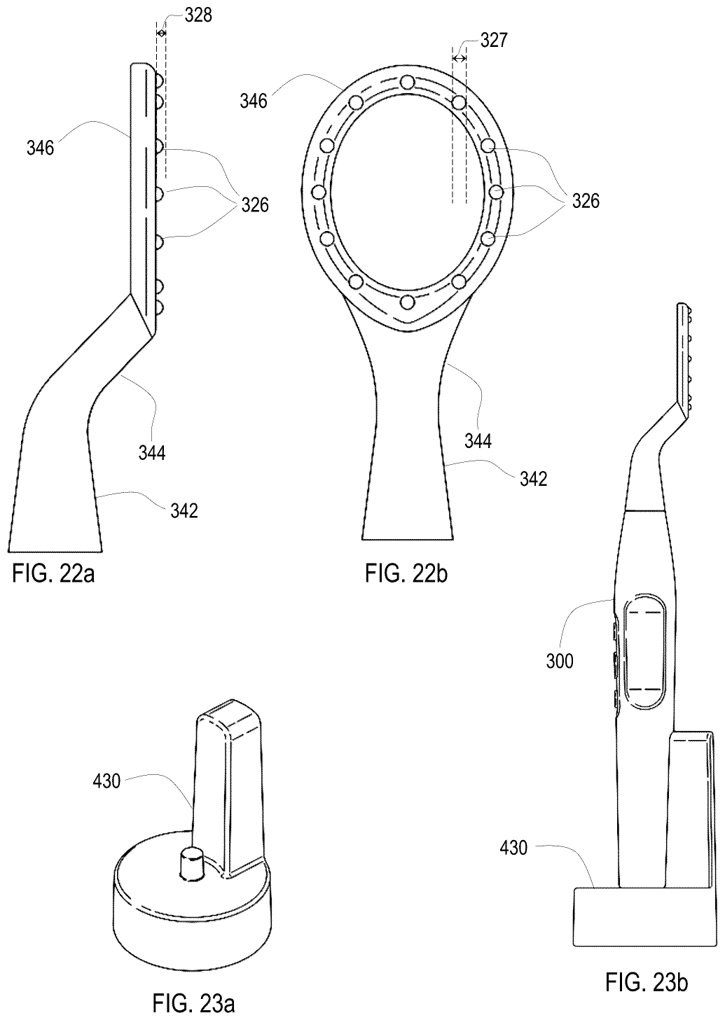

FIGS. 21a-21e shows a top view of various embodiments of the tip 340. In some embodiments, the contact element 346 is an open oval shape, as shown in FIG. 21b. In some embodiments, the contact element 346 is an open circular shape, as shown in FIG. 21c. In some embodiments, the contact element 346 has an opening and/or a discontinuity in the open geometric shape, as shown in FIGS. 21d and 21e. In some embodiments, the opening in the open geometric shape can be at any position of the geometric shape. In some embodiments, the opening in the open geometric shape is at about 90° when viewing the contact element 346 in a top view (as shown in FIG. 21d). In some embodiments, the opening in the open geometric shape is at about 0° when viewing the contact element 346 in a top view (as shown in FIG. 21d). In some embodiments, this opening and/or discontinuity in the open geometric shape allows for easier placement of needle on the patient's skin.

In some embodiments, the contact element 146/346 comprises a plurality of nodes 326 on the contact surface thereof. In some embodiments, the number of nodes in the plurality of nodes is in the range of from about 2 to 50, 2 to 24, 2 to 16, 2 to 12, 4 to 50, 4 to 24, 4 to 16, 4 to 12, 6 to 50, 6 to 24, 6 to 16, 6 to 12, 8 to 50, 8 to 24, 8 to 16, 8 to 12, 10 to 50, 10 to 24, 10 to 16, or 10 to 12.

The plurality of nodes 326 jut out from the surface of the contact element 146/346 on the side of the contact element 146/346 that will touch the patient. In some embodiments, the plurality of nodes 326 touches the skin or other part of the patient and the contact element 146/346 does not. In some embodiments, each of the nodes is oval, circular, or other geometrically shaped. In some embodiments, each of the nodes in the plurality of nodes are the same geometric shape. In some embodiments, one or more of the nodes in the plurality of nodes are a different geometric shape than the rest of the nodes in the plurality of nodes. In some embodiments, the nodes are rounded. In some embodiments, the nodes are flat.

In some embodiments, the plurality of nodes 326 result in an enhanced vibrational analgesia effect than that is produced in a similar device but without the plurality of nodes.

In some embodiments, the nodes 326 have a diameter 327 in the range of about 0.5 mm to 10 mm, 0.5 mm to 5 mm, 0.5 mm to 3 mm, 0.5 mm to 2 mm, 1 mm to 10 mm, 1 mm to 5 mm, 1 mm to 3 mm, 1 mm to 2 mm, 2 mm to 10 mm, 2 mm to 5 mm, 2 mm to 3 mm, 3 mm to 10 mm, or 3 mm to 5 mm. In some embodiments, the nodes 326 have height 328 in the range of about 0.25 mm to 5 mm, 0.25 mm to 3 mm, 0.25 mm to 2 mm, 0.25 mm to 1 mm, 0.5 mm to 5 mm, 0.5 mm to 3 mm, 0.5 mm to 2 mm, 0.5 mm to 1 mm, 1 mm to 5 mm, 1 mm to 3 mm, or 1 mm to 2 mm.

In some embodiments, there is no recharging port 172/372. Instead, the device according to one or more embodiments, as shown in FIGS. 23a and 23b, can be recharged via a charger stand 430 that charges the device 100/300 by using inductive current. In these embodiments, the battery 170/370 is configured to recharge via indicative current.

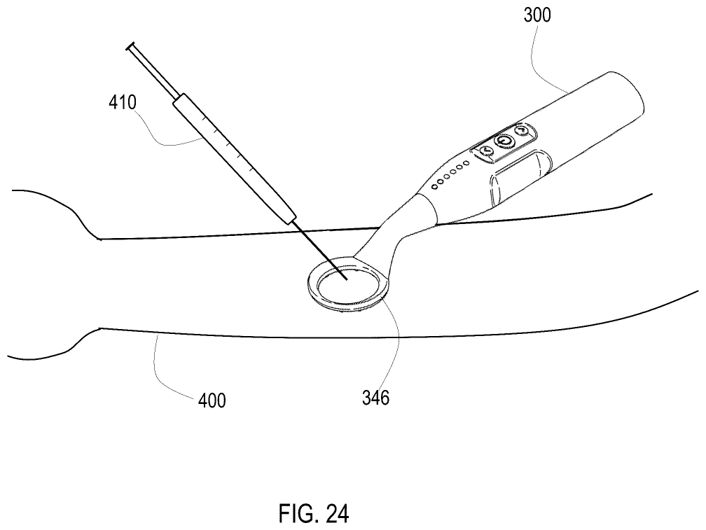

FIG. 24 shows the device 300 of one or more embodiments in use. The contact element 346 of device 300 will be positioned on an area of subject's arm 400. The vibratory motor 320 will be turned on. This will produce vibrations that are transferred from the contact element 346 to the subject's arm 400 in an anesthesia zone. The contact element 346 is maintained on the arm 400 for a time period prior to and during an application of a needle 410. In some embodiments, as shown in FIG. 24, the needle is injected into an area of the skin that is within the open geometric shaped contact element. The vibrations produce a vibrational analgesia effect on the arm 400 so as to reduce pain in the subject.

In some embodiments, there is no hinge and the proximal section 142/342 and the distal section 144/344 of tip 140/340 are one continuous element. In these embodiments where there is no hinge, the angle between the longitudinal axis of the hollow body 152/352 and the longitudinal axis of the distal section of the tip 156/356 is in the range of from about 0° to 90°, 0° to 80°, 0° to 70°, 0° to 60°, 0° to 50°, 0° to 40°, 0° to 30°, 0° to 20°, 0° to 10°, 10° to 90°, 10° to 80°, 10° to 70°, 10° to 60°, 10° to 50°, 10° to 40°, 10° to 30°, 10° to 20°, 20° to 90°, 20° to 80°, 20° to 70°, 20° to 60°, 20° to 50°, 20° to 40°, 20° to 30°, 30° to 90°, 30° to 80°, 30° to 70°, 30° to 60°, 30° to 50°, 30° to 40°, 40° to 90°, 40° to 80°, 40° to 70°, 40° to 60°, 40° to 50°, 50° to 90°, 50° to 80°, 50° to 70°, 50° to 60°, 60° to 90°, 60° to 80°, 60° to 70°, 70° to 90°, 70° to 80°, or 80° to 90°. In some embodiments, the angle between the longitudinal axis of the hollow body 152/352 and the longitudinal axis of the distal section of the tip 156/356 is about 45°. In some embodiments, the angle between the longitudinal axis of the hollow body 152/352 and the longitudinal axis of the distal section of the tip 156/356 is about 0°, 10°, 20°, 30°, 40°, 45°, 50°, 60°, 70°, 80°, or 90°.

In some embodiments, there is a hinge coupled to the proximal section of the tip 142/342 and the distal section of the tip 144/344. In these embodiments where there is a hinge, the angle between the longitudinal axis of the hollow body 152/352 and the longitudinal axis of the distal section of the tip 156 is in the range of from about 0° to 90°, 0° to 80°, 0° to 70°, 0° to 60°, 0° to 50°, 0° to 40°, 0° to 30°, 0° to 20°, 0° to 10°, 10° to 90°, 10° to 80°, 10° to 70°, 10° to 60°, 10° to 50°, 10° to 40°, 10° to 30°, 10° to 20°, 20° to 90°, 20° to 80°, 20° to 70°, 20° to 60°, 20° to 50°, 20° to 40°, 20° to 30°, 30° to 90°, 30° to 80°, 30° to 70°, 30° to 60°, 30° to 50°, 30° to 40°, 40° to 90°, 40° to 80°, 40° to 70°, 40° to 60°, 40° to 50°, 50° to 90°, 50° to 80°, 50° to 70°, 50° to 60°, 60° to 90°, 60° to 80°, 60° to 70°, 70° to 90°, 70° to 80°, or 80° to 90°. In some embodiments, the angle between the longitudinal axis of the hollow body 152/352 and the longitudinal axis of the distal section of the tip 156/356 is about 45°.

In some embodiments, the vibratory motor transmits vibrations at a frequency in the range of from about 2.5 Hz to 1000 Hz, 2.5 Hz to 750 Hz, 2.5 Hz to 550 Hz, 2.5 Hz to 500 Hz, 2.5 Hz to 300 Hz, 2.5 Hz to 250 Hz, 2.5 Hz to 200 Hz, 2.5 Hz to 150 Hz, 2.5 Hz to 100 Hz, 2.5 Hz to 50 Hz, 2.5 Hz to 25 Hz, 5 Hz to 1000 Hz, 5 Hz to 750 Hz, 5 Hz to 550 Hz, 5 Hz to 500 Hz, 5 Hz to 300 Hz, 5 Hz to 250 Hz, 5 Hz to 200 Hz, 5 Hz to 150 Hz, 5 Hz to 100 Hz, 5 Hz to 50 Hz, 5 Hz to 25 Hz, 25 Hz to 1000 Hz, 25 Hz to 750 Hz, 25 Hz to 550 Hz, 25 Hz to 500 Hz, 25 Hz to 300 Hz, 25 Hz to 250 Hz, 25 Hz to 200 Hz, 25 Hz to 150 Hz, 25 Hz to 100 Hz, 25 Hz to 50 Hz, 50 Hz to 1000 Hz, 50 Hz to 750 Hz, 50 Hz to 550 Hz, 50 Hz to 500 Hz, 50 Hz to 300 Hz, 50 Hz to 250 Hz, 50 Hz to 200 Hz, 50 Hz to 150 Hz, 50 Hz to 100 Hz, 100 Hz to 1000 Hz, 100 Hz to 750 Hz, 100 Hz to 550 Hz, 100 Hz to 500 Hz, 100 Hz to 300 Hz, 100 Hz to 250 Hz, 100 Hz to 200 Hz, 100 Hz to 150 Hz, 150 Hz to 1000 Hz, 150 Hz to 750 Hz, 150 Hz to 550 Hz, 150 Hz to 500 Hz, 150 Hz to 300 Hz, 150 Hz to 250 Hz or 150 Hz to 200 Hz.

In some embodiments, the vibratory motor operates at a voltage in the range of from about 0.05 mV to 10V, 0.05 mV to 5 V, 0.05 mV to 3V, 0.05 mV to 2V, 0.05 mV to 1V, 0.5 mV to 10V, 0.5 mV to 5 V, 0.5 mV to 3V, 0.5 mV to 2V, 0.5 mV to 1V, 5 mV to 10V, 5 mV to 5 V, 5 mV to 3V, 5 mV to 2V, 5 mV to 1V, 50 mV to 10V, 50 mV to 5 V, 50 mV to 3V, 50 mV to 2V, 50 mV to 1V, 500 mV to 10V, 500 mV to 5 V, 500 mV to 3V, 500 mV to 2V, 500 mV to 1V, 1V to 10V, 1V to 5 V, 1V to 3V, or 1V to 2V.

Vibrational source 120/320 can be any conventional vibrational source or means for producing vibrations. Examples of suitable vibrational sources include elliptical flywheel motors, eccentric motors, and the like. The vibrational source transfers vibration to the patient at a sufficient level to produce vibrational analgesia. For example, providing vibrations of between about 2.5 Hz to 1000 Hz to the patient prior to and during the needle stick to produce a suitable level of vibrational analgesia.

Vibrational source 120/320 produces a single vibrational cycle, multiple vibrational cycles, or can be variable. In other words, vibrational source 120/320 can be a vibrational motor that operates at, for example, 100 Hz or, for another example, at 200 Hz. Alternatively, vibrational source 120/320 can be a vibrational motor that operates at two or more vibrational cycles, for example, 100 Hz and 200 Hz, and can be switched between vibrational cycles by a switch or other mechanism. Alternatively, vibrational source 120/320 can be a vibrational motor that operates at many different vibrational cycles along a continuum by using a potentiostatic switch or a variable dimmer switch, for example, vibrational source 120/320 can be varied continuously or step-wise between 2.5 Hz and 1000 Hz. In some embodiments, a variable dimmer switch or potentiostatic switch, such as switch 130 can be configured to control the frequency and/or voltage of the vibrations transmitted. In some embodiments, one or more vibrational toggle buttons 332, 334 can be configured to control the frequency and/or voltage of the vibrations transmitted.

In some embodiments, if the user still feels pain and/or discomfort after the vibratory motor 120/320 is turned on at a first frequency and/or voltage, and the contact element 146/346 is positioned on and maintained on an area of subject's arm 200/400, then the frequency and/or voltage at which the vibratory motor 120/320 is operating at can be increased to a second, higher, frequency and/or voltage, to provide more of a vibrational analgesia effect. This process can be repeated as many times as is necessary to reduce the amount of pain and or discomfort to a desired level. In some embodiments, the user is asked to rate the pain. In some embodiments, the user will rate the pain based on the Wong Baker Faces Pain Rating Scale of 0-10 will be used (see wongbakerfaces.org). In some embodiments, if the user still feels pain and/or discomfort after the vibratory motor 120/320 is turned on at a first frequency and/or voltage, based on their rating using the Wong Baker Faces Pain Rating Scale, the then the frequency and/or voltage at which the vibratory motor 120/320 is operating at can be increased to a second, higher, frequency and/or voltage, to provide more of a vibrational analgesia effect. This process can be repeated as many times as is necessary to reduce the amount of pain and or discomfort to a desired level.

In some embodiments, switch 130 can be an on/off switch, such as a toggle, lever, push-button, capacitance or other switch. This type of switch 130 would be practical with a single vibrational cycle motor. Alternatively, switch 130 can be a common three-way switch. This type of switch 130 would be practical with a double vibrational cycle motor. Alternatively, switch 130 can be a potentiostat or variable dimmer switch. This type of switch 130 would be practical with a vibrational motor that operates at many different vibrational cycles along a continuum.

In some embodiments, on/off button 330 can be a toggle, lever, push-button or other switch. In some embodiments, the one or more vibration toggle buttons 332, 334 can be a toggle, lever, push-button or other switch. In some embodiments, a user can engage the one or more vibration toggle buttons 332, 334 to change the frequency and/or voltage of the vibrations transmitted.

In some embodiments, the number of different vibrational levels that the device can operate at is in the range of from about 1 to 15, 1 to 10, 1 to 6, 1 to 5, 2 to 15, 2 to 10, 2 to 6, or 2 to 5. In some embodiments, the number of different vibrational levels that the device can operate at is about 4 or 6. In some embodiments, the user can change between the different vibrational levels by depressing or otherwise engaging vibration toggle buttons 332, 334.

In some embodiments, the open geometric-shaped contact element has an open polygon shape, an open elliptical shape, or an open pictograph shape. In some embodiments, the open polygon shape can be an open triangular shape, an open quadrilateral shape, an open pentagonal shape, an open hexagonal shape, an open heptagonal shape, an open octagonal shape, an open nonagonal shape, an open decagonal shape, or the like. In some embodiments, the open quadrilateral shape can be an open rectangular shape or an open square shape. In some embodiments, the open elliptical shape can be an open oval shape, an open circular shape, or the like. In some embodiments, the open circular shape can also be described as an annulus shape or a ring shape. In some embodiments, the open pictograph shape can be an open-heart shape, an open star shape, an open animal shape, or the like. In some embodiments, the open animal shape can be an open butterfly shape, an open bird shape, an open dog shape, an open cat shape, or the like.

In some embodiments, the open geometric-shaped contact element has an inner diameter 147/347 that is in the range of from about 1 cm to 20 cm, 1 cm to 18 cm, 1 cm to 16 cm, 1 cm to 14 cm, 1 cm to 12 cm, 1 cm to 10 cm, 1 cm to 8 cm, 1 cm to 6 cm, 1 cm to 4 cm, 1 cm to 2 cm, 2 cm to 20 cm, 2 cm to 18 cm, 2 cm to 16 cm, 2 cm to 14 cm, 2 cm to 12 cm, 2 cm to 10 cm, 2 cm to 8 cm, 2 cm to 6 cm, 2 cm to 4 cm, 4 cm to 20 cm, 4 cm to 18 cm, 4 cm to 16 cm, 4 cm to 14 cm, 4 cm to 12 cm, 4 cm to 10 cm, 4 cm to 8 cm, 4 cm to 6 cm, 6 cm to 20 cm, 6 cm to 18 cm, 6 cm to 16 cm, 6 cm to 14 cm, 6 cm to 12 cm, 6 cm to 10 cm, 6 cm to 8 cm, 8 cm to 20 cm, 8 cm to 18 cm, 8 cm to 16 cm, 8 cm to 14 cm, 8 cm to 12 cm, 8 cm to 10 cm, 10 cm to 20 cm, 10 cm to 18 cm, 10 cm to 16 cm, 10 cm to 14 cm, 10 cm to 12 cm, 12 cm to 20 cm, 12 cm to 18 cm, 12 cm to 16 cm, 12 cm to 14 cm, 14 cm to 20 cm, 14 cm to 18 cm, 14 cm to 16 cm, 16 cm to 20 cm, 16 cm to 18 cm, 18 cm to 20 cm.

In some embodiments, the open geometric-shaped contact element has an outer diameter 148/348 that is larger than the inner diameter 147/347 by an amount that is in the range of from about 0.5 mm to 100 mm, 0.5 mm to 75 mm, 0.5 mm to 50 mm, 0.5 mm to 30 mm, 1 mm to 100 mm, 1 mm to 75 mm, 1 mm to 50 mm, 1 mm to 30 mm, 5 mm to 100 mm, 5 mm to 75 mm, 5 mm to 50 mm, or 15 mm to 30 mm 0.5 mm to 20 mm, 0.5 mm to 15 mm, 0.5 mm to 10 mm, 0.5 mm to 5 mm, 1 mm to 20 mm, 1 mm to 15 mm, 1 mm to 10 mm, 1 mm to 5 mm, 5 mm to 20 mm, 5 mm to 15 mm, 5 mm to 10 mm, or 15 mm to 20 mm.

In some embodiments, the open geometric-shaped contact element has a height 145/345 that is in the range of from about 0.5 mm to 20 mm, 0.5 mm to 15 mm, 0.5 mm to 10 mm, 0.5 mm to 5 mm, 1 mm to 20 mm, 1 mm to 15 mm, 1 mm to 10 mm, 1 mm to 5 mm, 5 mm to 20 mm, 5 mm to 15 mm, 5 mm to 10 mm, or 15 mm to 20 mm.

As discussed above, in some embodiments, the tip 140/340 is disposable. In some embodiments, the tip 140/340 is not disposable. In these embodiments, the tip 140/340 would be routinely cleaned with alcohol or other disinfectant between uses. In some embodiments, where the tip 140/340 is not disposable, the tip 140/340 can be sterilized in an autoclave or other suitable means.

In some embodiments, not shown, the contact element is not open geometric shape, but instead a solid or closed geometric shape. In some embodiments, the solid geometric-shaped contact element has a solid polygon shape, a solid elliptical shape, or a solid pictograph shape. In some embodiments, the solid polygon shape can be a solid triangular shape, a solid quadrilateral shape, a solid pentagonal shape, a solid hexagonal shape, a solid heptagonal shape, a solid octagonal shape, a solid nonagonal shape, a solid decagonal shape, or the like. In some embodiments, the solid quadrilateral shape can be a solid rectangular shape or a solid square shape. In some embodiments, the solid elliptical shape can be a solid oval shape, a solid circular shape, or the like. In some embodiments, the solid pictograph shape can be a solid heart shape, a solid star shape, a solid animal shape, or the like. In some embodiments, the solid animal shape can be a solid butterfly shape, a solid bird shape, a solid dog shape, a solid cat shape, or the like. In these embodiments, the device is positioned on an area of the subject's arm. The vibratory motor is turned on to produce vibrations that are transferred from the contact element to the subject's arm in an anesthesia zone. The contact element is maintained on the arm for a time period prior to or prior to and during the application of a needle. In some embodiments, the solid geometric-shaped contact element is left in contact with the arm and a needle is injected into a location near the contact element. In some embodiments, the solid geometric-shaped contact element is removed from the arm after being in contact for a predetermined period of time and the needle is injected into the area of the arm that the contact element was just removed from.

In some embodiments, the one or more of the components of the device 100/300 can be made from stainless steel, aluminum alloy, zinc, plastic, silicone, polymers, or a combination thereof. In some embodiments, the one or more of the components of the device 100/300 can be metal plated, such as gold plated.

In some embodiments, the tip 140/340 can be made from stainless steel, aluminum alloy, zinc, plastic, silicone, polymers, or a combination thereof. In some embodiments, the tip 140/340 can be metal plated, such as gold plated.

In some embodiments, the hollow body 110/310 can be made from stainless steel, aluminum alloy, zinc, plastic, silicone, polymers, or a combination thereof. In some embodiments, the hollow body 110/310 can be metal plated, such as gold plated.

The contact element 146/346 is applied to the patient for a time period sufficient to initiate vibrational analgesia, which can be between about 0 seconds and several minutes or more depending on the patient. In some embodiments, the contact element 146/346 is applied to the patient for a period of about 5 to 1200 seconds, 5 to 1000 seconds, 5 to 800 seconds, 5 to 600 seconds, 5 to 400 seconds, 5 to 200 seconds, 5 to 100 seconds, 50 to 1200 seconds, 50 to 1000 seconds, 50 to 800 seconds, 50 to 600 seconds, 50 to 400 seconds, 50 to 200 seconds, 50 to 100 seconds, 100 to 1200 seconds, 100 to 1000 seconds, 100 to 800 seconds, 100 to 600 seconds, 100 to 400 seconds, 100 to 200 seconds, 200 to 1200 seconds, 200 to 1000 seconds, 200 to 800 seconds, 200 to 600 seconds, 200 to 400 seconds, 400 to 1200 seconds, 400 to 1000 seconds, 400 to 800 seconds, 400 to 600 seconds, 600 to 1200 seconds, 600 to 1000 seconds, 600 to 800 seconds, 800 to 1200 seconds, 800 to 1000 seconds, or 1000 to 1200 seconds prior to an application of a needle, and continuing during the needle stick to provide a suitable level of vibrational analgesia.

In one or more embodiments, the device can be used with a needle with a gauge in the range of about 7 gauge to 34 gauge, 7 gauge to 30 gauge, 7 gauge to 25 gauge, 10 gauge to 34 gauge, 10 gauge to 30 gauge, 10 gauge to 25 gauge, 15 gauge to 34 gauge, 15 gauge to 30 gauge, or 15 gauge to 25 gauge.

In some embodiments, the device can be configured for use on any area of a subject's body. For example, the device can be configured for use on a subject's arm, back, neck, leg, scalp, face, etc. In some embodiments, for example, on a large area of skin such as a large area on the back of a patient, a larger tip can be used that has a larger number of nodes.

### EXAMPLES

In the examples that follow, one or more subjects will rate their pain caused by contact with various hypodermic needles with and without using the vibration device according to one or more embodiments of the present invention. In the example that follow, contact with the needle refers to contacting the skin of the patient to poke them, but not pierce them, with the needle.

The Wong Baker Faces Pain Rating Scale of 0-10 will be used (see wongbakerfaces.org). In this scale, a rating of 0 means “no hurt”, a rating of 2 means “hurts a little bit, a rating of 4 means “hurts a little more”, a rating a 6 means “hurts even more”, a rating of 8 means “hurts whole lot” and a rating of 10 means “hurts worst”. This numerical scale will be presented on a piece of paper that also has face emojis representing the different states of pain on the scale. This piece of paper will be given to the subjects as a guide to rate their pain.

The subjects will range in age from 18-75 years old. contact will be near the flexor forearm (anterior and/or medial locations), approximately 6 cm distal to the antecubital fossa, herein referred to as the “contact site”.

### Examples 1-3

In Examples 1 through 3, below, the device 100, as further described above, will be used.

### Example 1

Two different gauged needles will be used, a 22-gauge needle and a 30-gauge needle.

The first part of the experiment will test the pain of the needle contact without the vibration device. The subject's arm will be contacted with the 22-gauge needle three (3) times at or near the injection site. After each of the three contacts, the subject will be asked to rate their pain on a scale of 0-10, using the Wong Baker Faces Pain Rating Scale. The subject's answers will be recorded and an average of the subject's pain rating will be calculated.

Then, the vibration device according to one or more embodiments of the present invention will be placed around the same general area of the arm, that is, around the contact site and turned on to a frequency of 150 Hz. The vibration device will be left in place for 5 seconds and then removed. Immediately after the device is removed, the subject's arm will be contacted with the 22-gauge needle three (3) times at or near the contact site. After each of the three contacts, the subject will be asked to rate their pain on a scale of 0-10, using the Wong Baker Faces Pain Rating Scale. The subject's answers will be recorded and an average of the subject's pain rating will be calculated.

Then, the vibration device according to one or more embodiments of the present invention will be placed around the same general area of the arm, that is, around the contact site and turned on to a frequency of 150 Hz. The vibration device will be left in place while the arm is contacted with the 22-gauge needle three (3) times again. After each of the three contacts, the subject will be asked to rate their pain on a scale of 0-10, using the Wong Baker Faces Pain Rating Scale. The subject's answers will be recorded and an average of the subject's pain rating will be calculated.

The process described above will then be repeated using a 30-gauge needle on the subject's other arm.

The results will show that using the vibrational device according to one or more embodiments of the present invention at the contact site will reduce the pain perceived by subjects by needles.

### Example 2

A 22-gauge needle will be used to test the effect of the vibrational frequency of the vibration device according to one or more embodiments of the present invention.

The vibrational device will be turned on to a frequency of 75 Hz. The vibration device will be left in place while the arm is contacted with the 22-gauge needle three (3) times at or near the contact site. After each of the three contacts, the subject will be asked to rate their pain on a scale of 0-10, using the Wong Baker Faces Pain Rating Scale. The subject's answers will be recorded and an average of the subject's pain rating will be calculated.

Then, the vibrational device will be turned on to a frequency of 175 Hz. The vibration device will be left in place while the arm is contacted with the 22-gauge needle three (3) times at or near the contact site. After each of the three contacts, the subject will be asked to rate their pain on a scale of 0-10, using the Wong Baker Faces Pain Rating Scale. The subject's answers will be recorded and an average of the subject's pain rating will be calculated.

### Example 3

A 22-gauge needle will be used to test the effect of the inner diameter of the vibration device according to one or more embodiments of the present invention.

A first vibration device will have an inner diameter of 2 cm. The vibrational device will be turned on to a frequency of 150 Hz. The vibration device will be left in place while the arm is contacted with the 22-gauge needle three (3) times at or near the contact site. After each of the three contacts, the subject will be asked to rate their pain on a scale of 0-10, using the Wong Baker Faces Pain Rating Scale. The subject's answers will be recorded and an average of the subject's pain rating will be calculated.

A second vibration device will have an inner diameter of 5 cm. The vibrational device will be turned on to a frequency of 150 Hz. The vibration device will be left in place while the arm is contacted with the 22-gauge needle three (3) times at or near the contact site. After each of the three contacts, the subject will be asked to rate their pain on a scale of 0-10, using the Wong Baker Faces Pain Rating Scale. The subject's answers will be recorded and an average of the subject's pain rating will be calculated.

### Examples 4-5

In Examples 4 through 5, below, the device 300, as further described above, was used.

### Example 4

The device 300 with 12 nodes was used. Three different gauged needles were used, an 18-gauge needle, a 25-gauge needle and a 30-gauge needle.

The vibration device according to one or more embodiments of the present invention was placed on the contact site and turned on to a voltage of about 2 V. The vibration device was left in place and after a few seconds, the arm was contacted with the 18-gauge needle three (3) times. After each of the three contacts, the subject was asked to rate their pain on a scale of 0-10, using the Wong Baker Faces Pain Rating Scale. The subject's answers were recorded and an average of the subject's pain rating was calculated.

The process described above was repeated using a 25-gauge needle and the 30-gauge needle.

This procedure was repeated on eight (8) other subjects, for a total of nine (9) subjects.

### Example 5

The device 300 with 20 nodes was used. Three different gauged needles were used, an 18-gauge needle, a 25-gauge needle and a 30-gauge needle.

The vibration device according to one or more embodiments of the present invention was placed on the contact site and turned on to a voltage of about 2 V. The vibration device was left in place and after a few seconds, the arm was contacted with the 18-gauge needle three (3) times. After each of the three contacts, the subject was asked to rate their pain on a scale of 0-10, using the Wong Baker Faces Pain Rating Scale. The subject's answers were recorded and an average of the subject's pain rating was calculated.

The process described above was repeated using a 25-gauge needle and the 30-gauge needle.

This procedure was repeated on eight (8) other subjects, for a total of nine (9) subjects.

The results of the experiments performed in Examples 4 and 5 are shown below.

TABLE 1
Pain rating using the Wong Baker Faces Pain Rating Scale

| | 12 nodes | 20 nodes |
|---|---|---|
| 18-gauge needle | 0.8 | 1.1 |
| 25-gauge needle | 1.1 | 1.2 |
| 30-gauge needle | 1.0 | 1.7 |

The above examples show that the device 300, as further explained above, reduced the pain felt by subjects by contact with needles of various gauges. The results indicate that the device 300 with 12 nodes was more effective than a device with 20 nodes.

In the disclosure here, the terms “vibrational analgesia” and “vibrational anesthesia” are used interchangeably.

It will be appreciated by people skilled in the art that the present disclosure is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the examples disclosed herein are not necessarily exclusive of each other and may be included in a variety of different combinations or configurations without departing from the scope and spirit of the disclosure. A variety of modifications and variations are possible in light of the above teachings without departing from the scope and spirit of the disclosure, which is limited only by the following claims.

1. A device for reducing pain in a subject, comprising:
a. a hollow body having at least two ends;
b. a vibratory motor mounted in the hollow body configured for transmitting vibrations of variable frequency and/or voltage;
c. a switch or a button on the body to turn the vibratory motor on and off;
d. a non-single use tip having
a proximal section removably coupled to one end of the body,
a distal section extending forwardly from the body, and
a contact element on the distal section,
wherein the contact element has an open geometric shape that is an annulus or open oval, and
e. a hinge that is coupled to the proximal and distal sections of the tip, the hinge being adjustably configured to change the angle between the longitudinal axis of the tip and the longitudinal axis of the hollow body.

2. (canceled)

3. The device of claim 1, wherein the tip is disposable.

4. The device of claim 1, wherein the hollow body has a longitudinal axis,
wherein the proximal section of the tip has a longitudinal axis that is parallel with the longitudinal axis of the hollow body
wherein the distal section of the tip has a longitudinal axis that is at an angle in the range of from 0° to 90° relative to the longitudinal axis of the hollow body, and
wherein the contact element has a longitudinal axis that is at an angle in the range of from 0° to 90° relative to the longitudinal axis of the hollow body.

5. (canceled)

6. The device of claim 1, wherein the vibratory motor transmits vibrations at a frequency in the range of from 2.5 Hz to 1000 Hz.

7. (canceled)

8. The device of claim 1, wherein the vibratory motor is turned on and off by the button and wherein the device further comprises one or more vibration toggle buttons to control the frequency and/or voltage of the vibrations transmitted at discrete vibrational levels.

9.-11. (canceled)

12. The device of claim 1, wherein the open geometric-shaped contact element has an inner diameter that is in the range of from 1 cm to 50 cm.

13. The device of claim 1, wherein the contact element is configured for transferring the vibrations from the motor to an anesthesia zone on an area of skin of the subject when the tip touches the skin, thereby producing a vibrational analgesia effect and reducing pain the zone.

14. The device of claim 1, wherein the contact element further comprises a plurality of nodes.

15. A method for reducing pain in a subject, comprising:
providing a device comprising:
a. a hollow body having at least two ends;
b. a vibratory motor mounted in the hollow body configured for transmitting vibrations of variable frequency and/or voltage;
c. a switch or a button on the body to turn the vibratory motor on and off;
d. a non-single use tip having
a proximal section removably coupled to one end of the body,
a distal section extending forwardly from the body, and
a contact element on the distal section,
wherein the contact element has open geometric shape that is an annulus or open oval, and
e. a hinge that is coupled to the proximal and distal sections of the tip, the hinge being adjustably configured to change the angle between the longitudinal axis of the tip and the longitudinal axis of the hollow body;
positioning the contact element on an anesthesia zone;
turning on the vibratory motor to produce vibrations from the motor that are transferred to the anesthesia zone; and
maintaining the contact element on the anesthesia zone for a time period prior to and during an application of a needle to the anesthesia zone,
whereby the vibrations are configured to produce a vibrational analgesia effect on the subject so as to reduce pain in the subject.

16. (canceled)

17. The method of claim 15, wherein the tip is disposable.

18. The method of claim 15, wherein the hollow body has a longitudinal axis,
wherein the proximal section of the tip has a longitudinal axis that is parallel with the longitudinal axis of the hollow body
wherein the distal section of the tip has a longitudinal axis that is at an angle in the range of from 0° to 90° relative to the longitudinal axis of the hollow body, and
wherein the contact element has a longitudinal axis that is an angle in the range of from 0° to 90° relative to the longitudinal axis of the hollow body.

19. (canceled)

20. The method of claim 15, wherein the vibratory motor transmits vibrations at a frequency in the range of from 2.5 Hz to 1000 Hz.

21.-25. (canceled)

26. The method of claim 15, wherein the open geometric-shaped contact element has an inner diameter that is in the range of from 1 cm to 50 cm.

27. The method of claim 15, wherein the contact element is maintained on the anesthesia zone for a time period in the range of from 5 to 1200 seconds.

28. The method of claim 15, wherein the contact element of the tip is configured for transferring the vibrations from the motor to an anesthesia zone on an area of skin of the subject when the tip touches the skin.

29. The method of claim 15, wherein the contact element further comprises a plurality of nodes.
